(19) United States
(12) Patent Application Publication
(10) Pub. No.: US 2024/0293674 A1
(43) Pub. Date: Sep. 5, 2024

(54) DETECTING AWAKE OR SLEEP STATUS AND RESPIRATION USING AN ACCELEROMETER AND A MAGNETOMETER

(71) Applicant: The Alfred E. Mann Foundation for Scientific Research, Valencia, CA (US)

(72) Inventors: Brian Michael SHELTON, Altadena, CA (US); Sahar ELYAHOODAYAN, Los Angeles, CA (US)

(21) Appl. No.: 18/593,327

(22) Filed: Mar. 1, 2024

Related U.S. Application Data
(60) Provisional application No. 63/488,049, filed on Mar. 2, 2023.

(51) Int. Cl. A61N 1/36

(52) U.S. Cl. CPC A61N 1/3611; A61N 1/36139

(57) ABSTRACT

The present disclosure generally relates to systems and methods for detecting awake or sleep states and/or monitoring respiratory events (e.g., inspiration and expiration) for a subject using measurements obtained from at least an accelerometer and a magnetometer, and methods of treating medical conditions related thereto (e.g., obstructive sleep apnea). Positional data generated by the accelerometer or magnetometer is then used by a controller to control the magnetometer and accelerometer according to different sampling rates and to reset signal filters based at least in part on a detection that a body orientation of the subject has changed after detecting that the subject is asleep.

### CROSS-REFERENCE TO RELATED APPLICATION

This application claims the benefit to U.S. Provisional Patent Application No. 63/488,049, entitled “DETECTING AWAKE OR SLEEP STATUS AND RESPIRATION USING AN ACCELEROMETER AND A MAGNETOMETER” and filed Mar. 2, 2023, the disclosure of which is hereby incorporated by reference in its entirety.

### TECHNICAL FIELD

The present disclosure generally relates to systems and methods for detecting inspiration and respiration of an individual using at least a magnetometer and accelerometer, and methods of treating medical conditions related thereto.

### BACKGROUND

Obstructive Sleep Apnea (OSA) is a sleep disorder involving obstruction of the upper airway during sleep. The obstruction of the upper airway may be caused by the collapse of or increase in the resistance of the pharyngeal airway, often resulting from tongue obstruction. The obstruction of the upper airway may be caused by reduced genioglossus muscle activity during the deeper states of Non-Rapid Eye Movement (NREM) sleep. Obstruction of the upper airway may cause breathing to pause during sleep. Cessation of breathing may cause a decrease in the blood oxygen saturation level, which may eventually be corrected when the person wakes up and resumes breathing. The long-term effects of OSA include high blood pressure, heart failure, strokes, diabetes, headaches, and general daytime sleepiness and memory loss, among other symptoms.

OSA is extremely common, and may have a prevalence similar to diabetes or asthma. Over 100 million people worldwide suffer from OSA, with about 25% of those people being treated. Continuous Positive Airway Pressure (CPAP) is a conventional therapy for people who suffer from OSA. More than five million patients own a CPAP machine in North America, but many do not comply with use of these machines because they cover the mouth and nose and, hence, are cumbersome and uncomfortable.

Neurostimulators may be used to open the upper airway as a treatment for alleviating apneic events. Such therapy may involve stimulating the nerve fascicles of the hypoglossal nerve (HGN) that innervate the intrinsic and extrinsic muscles of the tongue in a manner that prevents retraction of the tongue which would otherwise close the upper airway during the inspiration period of the respiratory cycle. ImThera Medical is currently in Food and Drug Administration (FDA) clinical trials for a stimulator system that is used to stimulate the trunk of the hypoglossal nerve stimulation (HGN) with a nerve cuff electrode. The stimulation system does not provide a sensor or sensing, and therefore, the stimulation delivered to the HGN trunk is not synchronized to the respiratory cycle. Thus, the tongue and other muscles that are innervated by nerve fascicles of the HGN trunk are stimulated irrespective of the respiratory cycle.

### BRIEF SUMMARY OF EXEMPLARY ASPECTS OF THE DISCLOSURE

Ideally, a system for treating OSA should be able to detect inspiration and respiration in the subject. For instance, stimulation should be applied during inspiration, which in turn saves energy and prevents muscle fatigue as compared with indiscriminately applying stimulation regardless of inspiration and respiration events. Accordingly, the detection of respiration uses a certain amount of energy when the detection is performed through pressure, acceleration, rotation, sound, or some other means. Therefore, it is generally desirable to reduce the frequency of measurements or shut off measurements when not needed in order to converse energy. In addition, it may also be desirable to start and stop stimulation automatically. This may allow the subject using the OSA system to essentially forget about their sleep apnea and also eliminate the problem of forgetting to start stimulation before going to bed.

Moreover, there is a need in the art for OSA stimulation systems that can detect if a subject is awake or sleep and/or detect inspiration and expiration using sensors that obtain as few measurements as possible. For instance, stimulation systems cannot solely rely on a body orientation of a subject to detect if the subject is sleeping or awake. As a non-limiting example, a subject may be participating in activities, such as swimming or reading a book, that involve lying down for an extended period of time without the subject being in a sleep state. Therefore, it would be necessary to perform another type of check of the subject to determine if the subject is actually asleep or participating in an activity that involves laying down. Unfortunately, current OSA stimulation systems making this type of periodicity check will most likely use frequent sensor measurements of a subject and most likely use more energy.

The present disclosure addresses these and other shortcomings by providing systems and methods that can accurately detect and/or monitor whether a subject is asleep or wake and/or detect inspiration and expiration using a magnetometer in conjunction with an accelerometer in as few measurements as possible. In some aspects, an accelerometer may detect the body orientation (e.g., vertical, prone, supine, lateral, or the like) of the subject and a magnetometer may add the ability to detect a body orientation direction (e.g., a chest rotating upward, downward, outward, onward, or the like) of the subject. For example, measurement data from a magnetometer may be used to determine motion artifact or significant torso movement from the subject which is helpful to rule-out sleep by using fewer sensor measurements as compared to using other types of sensor such as a gyroscope. Such systems may advantageously be used to control and adjust a sampling rate of at least the magnetometer and the accelerometer based on a body orientation of the subject or whether the subject is asleep or wake in order to reduce power consumption and prevent muscle fatigue, among other benefits which will become apparent in view of the following descriptions and accompanying figures. For example, the present disclosure provides systems and methods where a magnetometer may be used to rule out sleep, helping to extend the battery recharge interval of the OSA systems.

The magnetometer and the accelerometer are designed to detect chest and/or abdominal movement and orientation for a subject, which is in turn processed by a controller communicatively linked to the magnetometer and the accelerometer, in order to accurately determine a body orientation and/or body direction of the subject. This allows for scenarios where a magnetometer can be used during a sleep period of the subject to determine whether the subject is awake or asleep. Another advantage may be using an opportunity when the subject is asleep as an ideal time to reset signal processing filters that are used for extracting a respiration signal from an accelerometer, gyroscope, microphone, or some other respiration sensor. This allows the system to use different filters and extraction techniques when the subject is at rest and during motion artifact to extract different features.

The following presents a simplified summary of several exemplary embodiments in order to provide a basic understanding of the inventions described herein. This summary is not intended as an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

In a first general aspect, the disclosure provides a system for detecting inspiration and respiration of a subject that includes an accelerometer configured to sense body status of the subject at a first sampling rate and generate a first accelerometer signal; a controller coupled to the accelerometer and configured to control sampling rates of the accelerometer; and a first filter comprising an envelope detector and a low pass filter. The first filter may be configured to process the first accelerometer signal and generate a body orientation signal corresponding to a body orientation of the subject. The controller may be further configured to determine if the body orientation of the subject is recumbent based on the body orientation signal.

In some aspects, the system further includes a magnetometer coupled to the controller and configured to sense the body status of the subject and generate a magnetometer signal at the first sampling rate. The first filter may be further configured to process the magnetometer signal and generate a body direction signal corresponding to a body direction of the subject. The controller may be further configured to determine if the subject is motionless based on the body direction signal.

In some aspects, the accelerometer is further configured to sense body movement at a second sampling rate and generate a second accelerometer signal. The second sampling rate may be greater than the first sampling rate. In some aspects, the system further comprises a second filter comprising a bandpass filter. The second filter may be configured to process the second accelerometer signal and to generate a respiration signal corresponding to the respiration of the subject. The controller is further configured to determine if the respiration signal is regular.

In some aspects, the controller is further configured to determine a body heading signal of the subject based at least in part on processing the body orientation signal and the body direction signal.

In some aspects, the controller is further configured to determine that the subject is asleep based at least in part on processing the respiration signal and at least one of the body orientation signal, the body direction signal, and the body heading signal.

In some aspects, the controller is further configured to determine inspiration and expiration phases of the respiration of the subject based at least in part on processing the respiration signal and at least one of the body orientation signal, the body direction signal, and the body heading signal.

In some aspects, the system further includes a stimulation system for generating nerve stimulation signals, and a lead coupled to the stimulation system and comprising a neural interface at its distal end. The controller being coupled to the stimulation system and further configured to control generating the nerve stimulation signals during the inspiration phase of the respiration of the subject.

In some aspects, the first sampling rate is one sample in a range of every 2 to 10 seconds.

In some aspects, the second sampling rate is in a range of 1 to 10 Hz.

In some aspects, the low pass filter has a cutoff frequency in a range of 0.1 to 2.0 Hz.

In some aspects, the bandpass filter has a passband within a range of 0.1 to 2.0 Hz.

In some aspects, the system further includes a gyroscope coupled to the controller and configured to sense body direction change and generate a gyroscope signal at the second sampling rate. The second filter may be further configured to process the gyroscope signal to provide a body direction change signal corresponding to the body direction change of the subject. The controller may be further configured to determine that the respiration of the subject is regular based on the body direction change signal.

In some aspects, the controller is further configured to determine that the subject is vertical based on at least one of the body orientation signal, body direction signal, and body heading signal. The first and second filters comprise digital filters having internal initial states. The controller may be further configured to reset the internal states of the first and second filters to initial states.

In a second general aspect, the disclosure provides methods of determining a respiration of a subject using any one of the systems described herein. For example, in some aspects the disclosure provides a device for use in treating sleep apnea, comprising any of the systems described herein. It is understood that a method of treating sleep apnea may comprise any combination of the steps or parameters described herein. For example, a method of treating OSA in a subject may comprise: sensing, using an accelerometer, body status of the subject at a first sampling rate in a range of every 2 to 10 seconds; generating, using the accelerometer, a first accelerometer signal; processing the first accelerometer signal with a first filter and generating a body orientation signal, wherein the first filter comprises an envelope detector and a low pass filter, wherein the low pass filter comprises a cutoff frequency in a range of 0.1 to 2.0 Hz; and determining if a body orientation is recumbent based on the body orientation signal.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a system for treating OSA for a human subject based upon sensor data. In this example, the system includes at least a magnetometer and an accelerometer integrated into the house of an OSA stimulation system.

FIG. 2 is a block diagram illustrating an exemplary embodiment of a system for treating OSA.

FIG. 3 is a conceptual flowchart summarizing a method for using a magnetometer and an accelerometer to detect whether a subject is awake or asleep and to determine the respiration of the subject, using the systems and methods described herein. The flowchart consists of at least a magnetometer and an accelerometer configured to obtain measurements according to using different sampling rates.

FIG. 4 is a conceptual flowchart summarizing a method for resetting signal filters based on using a magnetometer and an accelerometer to detect whether a subject is awake or asleep using the systems described herein, and optionally adjusting OSA stimulation based on the same.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

As noted above, the present disclosure is generally directed to systems and methods for detecting and/or monitoring whether a subject is asleep or awake and/or detecting inspiration and respiration using a magnetometer and accelerometer, and methods of treating medical conditions related thereto. When detecting whether a subject is asleep or awake or detecting respiration events using sensors, it is generally desirable to make as few measurements as possible to conserve energy in the system.

Current systems for the detection of a wake/sleep state of a subject suffer from various drawbacks. For example, the detection of a wake and sleep state of a subject typically uses frequent sensor measurements to determine that a subject is actually sleeping and not just participating in an activity that involves laying down. In these situations, sensors will need to continuously check measurements or check the subject by other means (e.g., periodicity, depth, or respiration) to determine if a subject is actually asleep. These frequent measurements will most likely use more energy due to faster sampling rates which will raise average energy consumption. Moreover, such systems may not accurately distinguish inspiration from expiration since an expansion of a chest of the subject in 3D space during inspiration is dependent on subject orientation and breathing style.

In contrast, the OSA system described herein simplify and improve on existing designs by incorporating additional sensor functionality into one or more components of the system. A system according to the disclosure may utilize at least a magnetometer and an accelerometer to detect whether a subject is awake or asleep and/or respiration events. For instance, such systems may utilize a magnetometer to determine a direction a subject is laying in and also during a subject's sleep period for determining motion artifact (e.g., gross subject movement). The system may also apply enveloping and using a smoothing function in order to detect motion artifact. This in turn may be used to reset filters that are used for extracting respiration signals from an accelerometer, gyroscope, microphone, or the like.

In some aspects, the magnetometer and the accelerometer may determine a respiration event and subject motion using different sampling rates. In some cases, detecting a motion artifact in subject orientation is a relatively infrequent activity and uses infrequent sampling (e.g., every 2 to 10 seconds). By contrast, respiration is a relatively quick activity and uses frequent sampling (e.g., 1 to 10 Hz). More frequent sampling rates uses more energy. In that sense, adjusting the accelerometer or the magnetometer to a lower sampling rate will reduce the average energy consumption of the system. In addition, if really low sampling rates (e.g., less than 1 Hz) are needed, then the system may simply turn the sensor on for a single measurement when it is needed. The system described herein are also particularly advantageous with respect to detecting wake and sleep states of a subject and reducing the frequency of measurement based on sampling rates. As explained above, earlier systems have used periodicity and depth of respiration to determine if a subject is asleep using a gyroscope. However, these earlier systems use more energy and will use more frequent sensor measurements leading to larger current draw for the OSA system.

Systems according to the disclosure may also be configured to distinguish inspiration from expiration of a subject by using a magnetometer to detect which way the sensor is heading in reference to the subject's chest. This is helpful as an expansion of the chest in 3D space during inspiration is dependent on subject orientation and breathing style. A breathing style may vary from subject to subject, and may even vary throughout the sleep cycle of a subject. Other factors may include whether the subject is an abdominal or chest breather, and how a subject's chest rotates during respiration. Accordingly, the accelerometer and magnetometer measurement data may be fused to obtain heading information used to enable the system to accurately distinguish inspiration from expiration in different orientations of the subject.

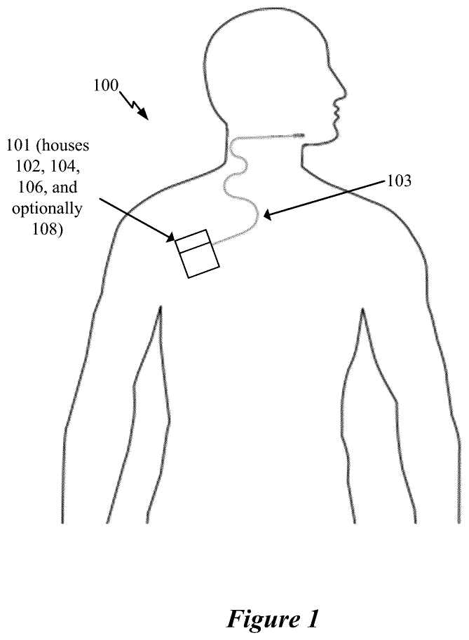

FIG. 1 is a diagram illustrating an embodiment of a system 100 for treating OSA for a human subject based upon sensor data. In this example, the system 100 comprises an implanted controller 102, a magnetometer 104, and an accelerometer 106 integrated into the housing 101 of the system 100. In some aspects, the system 100 also optionally comprises a gyroscope 108 integrated into the housing 101 of the system 100. One or more electrodes 103 extend from the housing 101 of the system 100 to deliver stimulation to one or more nerves which innervate an upper airway muscle of the subject.

In this example, the system 100 comprises at least two sensors—a magnetometer 104 and an accelerometer 106 configured to obtain measurement data related to movement and body orientation of a subject during the inspiration and expiration stages of a respiratory cycle (e.g., movement of the thoracic or abdominal cavity of the subject). In some aspects, measurement data obtained from the magnetometer 104 may be used in conjunction with measurement data obtained from the accelerometer 106 to improve the ability of the system 100 to detect if the subject is asleep or awake. In some aspects, measurement data obtained from the accelerometer 106 may be used to determine if the subject is vertical, prone, supine, or laying on his/her side. In some aspects, measurement data from the magnetometer 104 adds the ability to determine in which direction a subject is lying. In previous systems, measurement data obtained from a Micro-Electro-Mechanical System (MEMS) gyroscope may be used to determine what direction a subject is lying, however, using a MEMS gyroscope (such as gyroscope 108) typically takes about five times the average current as compared with using a magnetometer to determine the direction of a subject. When detecting wake and sleep state, it is preferable for a system to make as few measurements as possible and to keep the average current draw as low as possible to conserve energy in the system 100.

For instance, a subject may participate in activities that involve lying down for an extended period of time without the subject actually being asleep. An example of this type of activity is swimming. Accordingly, while the subject is swimming, a periodic check using a magnetometer 104 may indicate that the subject is drastically changing orientation (˜180 degrees) every minute. This periodic check of body orientation would help differentiate between a subject sleeping and the subject performing another activity that uses laying down or being horizontal. Thus, it is not difficult, nor would it use much energy for a system to check the subject a few times each minute to determine if the orientation of the subject has changed.

Another activity that a subject may perform while laying down and awake may be activities such as reading a book, watching television, or stretching. Therefore, it would be helpful to check by some other means (e.g., periodicity, depth of respiration, heart rate, or heart rate variability) whether the subject is asleep. However, these types of sleep indicator checks will most likely use different types of sensors and much more frequent sensor measurements, which both contribute to requiring more energy. Thus, utilizing a magnetometer to rule out sleep offers the opportunity to extend battery recharge interval.

In this exemplary embodiment, system 100 comprises a housing 101 that includes at least the magnetometer 104, the accelerometer 106, and a controller 102 configured to handle signal processing and storage and operation of the OSA stimulation system. System 100 may be an OSA stimulation system that further includes one or more electrodes 103 to deliver stimulation to one or more nerves which innervate an upper airway muscle of the subject. As described herein, controller 102 may be configured to control the sampling rate of at least the magnetometer 104 and the accelerometer 106 based on the body orientation of the subject or whether the subject is awake of asleep. In some aspects, the controller 102 may be configured to reset signal filters based at least in part on a detection that the body orientation of the subject has changed by using the accelerometer controlled according to a slower sampling rate to detect a respiration event and the magnetometer controlled according to a faster sampling rate than the accelerometer to detect movement of the subject after detecting that the subject is asleep.

In this example, system 100 utilizes at least measurement data obtained from the magnetometer 104 and the accelerometer 106 to improve an implant's ability to detect if the subject is asleep or awake. The inclusion of the magnetometer 104 within the housing 101 of system 100 is advantageous in that it reduces the frequency of sensor measurements needed to determine whether a subject is asleep or awake. Indeed, as noted above, prior systems which utilize measurement data from a MEMS gyroscope used to detect whether a subject is awake or asleep typically use more frequent measurements which may use more energy from the system and average a higher current draw. For instance, a MEMS gyroscope typically takes about five times the average current as compared with using an magnetometer. In addition, magnetometer 104 may be used during a subject's sleep period to detect motion artifact (e.g., or gross subject motion). For instance, gross subject motion may be defined as tasks involving movements of the large muscles of the arms, legs, and torso, as well as whole-body movements. Accordingly, these types of gross subject motion are indicative of non-sleeping activities such as walking, crawling, jumping, or the like. These types of detections may be used to reset signal filters that are used for extracting a respiration signal from an accelerometer, gyroscope, microphone, or some other respiration sensor. The signal filters are used to remove heartbeat effects and low-DC components such as subject movement. In some aspects, signal filters may also be used in sleep state or sleep stage detection. Thus, using a magnetometer 104 to rule out sleep offers an opportunity to extend the battery recharge interval for the system 100.

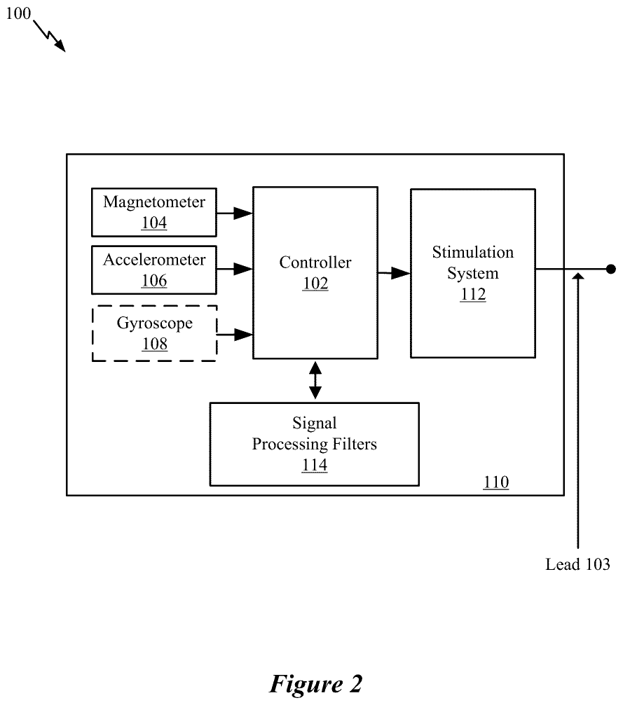

FIG. 2 is a block diagram of an exemplary embodiment of a system 100 for treating OSA. In this example, an implantable stimulator 110 includes a controller 102, a magnetometer 104, an accelerometer 106, an optional gyroscope 108, signal processing filters 114, and a stimulation system 112 (e.g., an IPG), and an electrode 103 which may be used to stimulate a respiratory system muscle of a subject.

Magnetometer 104 may be configured to obtain measurement data and this measurement data can be processed in turn by the controller 102 to determine a body orientation of a subject. The body orientation may be used by controller 102 to determine if there is a change in body orientation within a predefined time period (e.g., three minutes) for a subject, which in turn may be used to determine that the subject is not asleep. In some aspects, the lack of orientation change may be used by the controller 102 to cause a gyroscope 108 to begin periodic gyroscope measurements. For instance, 20 Hz measurements may be used to detect respiration. In some aspects, the lack of orientation change may also be used by controller 102 to cause the gyroscope 108 to begin more frequent accelerometer measurements (e.g., a sampling rate of greater than or equal to 10 Hz). In some aspects, the gyroscope may be set to a faster rate to determine respiration. In some aspects, an orientation change during a sleep period of a subject may be used to reset signal processing filters 114. In some aspects, the orientation change may be used by controller 102 to re-calibration a subject orientation with respect to the earth's magnetic field.

In addition, magnetometer 104 may be used to distinguish inspiration from expiration of a subject by determining which direction the sensor is heading in reference to the subject's chest. An absolute orientation can be determined by using the coordinate frame obtained from the accelerometer 106 relative to the north, east, and down frame obtained from the magnetometer 104. This is helpful as the expansion of the subject's chest in three-dimensional (3D) space during inspiration is dependent on subject orientation and breathing style. The breathing style may vary from subject to subject and even throughout a sleep cycle of an individual subject. In addition, the breathing style may also be affected by whether the subject is an abdominal or chest breather, and how the chest of a subject rotates (e.g., upward, downward, outward, or onward) during respiration.

The accelerometer 106 may be configured to obtain accelerometer data which can be processed in turn by the controller 102 to determine a body direction of the subject. Sensor fusion of the magnetometer and accelerometer enables re-calibration of subject orientation with respect to the earth's magnetic field. In particular, measurement data from the magnetometer 104 and the accelerometer 106 may be fused by the controller 102 to generate heading information used to help the controller 102 to distinguish inspiration from expiration in different orientations.

Controller 102 may also be configured to detect whether the subject is asleep or awake based at least in part on the body orientation or the direction of the subject. The controller 102 may also be configured to control the magnetometer 104 and the accelerometer 106 according to a first sampling rate (e.g., roughly every 2 to 10 seconds) or a second sampling rate (e.g., 10 Hz) based at least in part on the body orientation or a detection of whether the subject is asleep or awake. It is noted that different sampling rates are used for determining respiration and motion of the subject. For instance, large torso movement is a change in subject orientation and uses infrequent (or slower) sampling at a first sampling rate (e.g., in the range of every 2 to 10 seconds) because a more infrequent check using a magnetometer would indicate that the subject is drastically changing body orientation. In contrast, respiration is a relatively quick activity and, thus, requires a more frequent (or faster) sampling rate at a second sampling rate that is quicker than the first sampling rate (e.g., at 1 to 10 Hz) because frequent checks using the magnetometer are needed to indicate that the specific inspiration and respiration motions while the subject is breathing. Faster sampling rates use more energy. Thus, setting the accelerometer, magnetometer, or gyroscope to a lowest sampling rate needed will reduce the average energy consumption of the system. Accordingly, if even lower sampling rates are needed (e.g., less than 1 Hz), then this may be accomplished by turning on the sensor for a single measurement when it is needed.

The signal processing filters 114 are filters used for extracting the respiration and body status signals from an accelerometer 106, gyroscope 108, microphone, or some other respiration sensor. In some aspect, the signal processing filters 114 may be used to remove heartbeat effects and low-DC components (e.g., subject movements). In some aspects, the filters used during respiration are typically between 0.1 to 2 Hz. The controller 102 may further be configured to: after detecting that the subject is asleep, reset signal processing filters 114 based at least in part on a detection that the body orientation has changed by using the accelerometer 106 controlled according to the second sampling rate to detect a respiration event (e.g., inspiration or respiration) and the magnetometer 104 controlled according to a first sampling rate to detect movement of the subject

System 100 may be configured to deliver stimulation to a nerve innervating the upper airway of the subject through the electrode 103 implanted proximate to the nerve. In some embodiments, the nerve is the hypoglossal nerve. In some embodiments, the upper airway muscle comprises the genioglossus, the geniohyoid, or some combination thereof. When the nerve is stimulated, it activates the upper airway muscle, thereby preventing or alleviating obstructive apneic events. In some embodiments, the stimulation system 112 applies stimulation to the nerve with an intensity sufficient to promote tonus in the upper airway muscle. In some embodiments, the stimulation system 112 applies stimulation to the nerve with an intensity sufficient to cause bulk muscle movement in the upper airway muscle. The stimulation system 112 is coupled to controller 102. Controller 102 receives the measurement data from the magnetometer 104, the accelerometer 106, and/or optionally the gyroscope 108, and controls when the stimulation system 112 applies stimulation. In some embodiments, controller 102 can control the intensity of the stimulation applied by the stimulation system 112. In some embodiments, the stimulation system 112 may apply different intensities of stimulation by changing the amplitude, the pulse width, or the frequency of the stimulation. In some aspects, controller 102 controls the amplitude, the pulse width, or the frequency of the stimulation applied by the stimulation system 112.

The implantable stimulator 110 may be configured to receive sensory data from the magnetometer 104, the accelerometer 106, and/or optionally the gyroscope 108 (e.g., positioned within a housing containing the IPG) and to apply stimulation therapy to the subject based on the measurement data received from the magnetometer 104, the accelerometer 106, and/or optionally the gyroscope 108. Apneic events can be detected by determining that the regular respiratory pattern has become irregular for a number of cycles (e.g., at least 1, 2, 3, 4, 5, 6, 7, 8, 9, or 10 cycles). Waveforms and parameters indicative of an irregular respiratory pattern are disclosed, e.g., in U.S. Pat. Nos. 5,540,731 and 8,938,299, the entire contents of which are incorporated herein by reference. Positional data obtained from the magnetometer 104, the accelerometer 106, and/or optionally the gyroscope 108 may be used by the controller 102 for apnea detection (e.g., heart rate rhythm and variability), providing potential benefits such as increased accuracy with respect to the administration of stimulation as a treatment for OSA.

In some aspects, the positional data generated by the magnetometer 104, the accelerometer 106, and/or optionally the gyroscope 108 may need to be subject to signal processing via one or more analog or digital filters in order to generate useful information. For example, the magnetometer 104, the accelerometer 106, and/or optionally the gyroscope 108 may pick up extraneous movement signals (e.g., heartbeat and snoring sounds), that may need to be filtered out to selectively identify the soft and low-pitched sounds arising from inspiration and expiration. In some aspects, digital filters will be preferable. However, implementations that perform analog filtering may be simpler and will often be more energy efficient.

In some aspects, positional data from one of the magnetometer 104, the accelerometer 106, and/or optionally the gyroscope 108 may be filtered with a BPF. For example, a band pass filter with a passband in the range of 0.1 to 2.0 Hz. The low pass filter may be designed to pass respiration frequencies, while blocking cardiac frequencies and other sources of noise. For example, an upper cut-off frequency of 0.1 to 2 Hz would typically suffice to separate noise from the respiration signal. In some aspects, this filter could be an adaptive filter. A HPF may optionally be applied to eliminate any DC offset, with a high pass cut-off frequency of 0.05 to 0.1 Hz typically being sufficient in this regard.

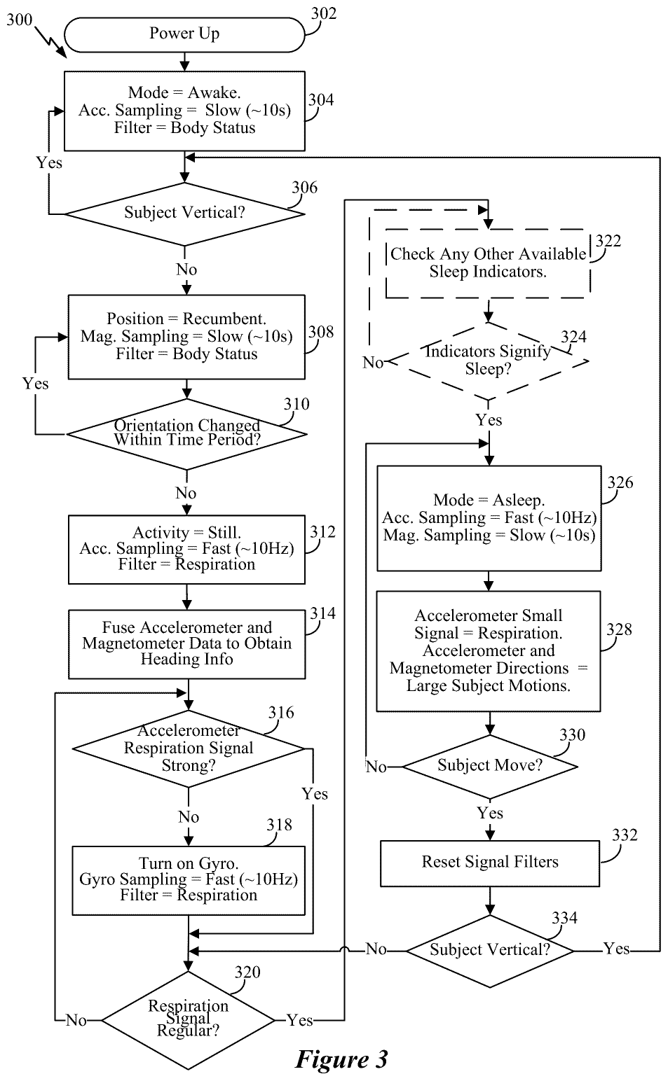

FIG. 3 is a conceptual flowchart summarizing a method 300 for using a magnetometer and an accelerometer to detect whether a subject is awake or asleep and to detect respiration of a subject, using the systems and methods described herein. Specifically, the method 300 may include controlling the sampling rate of the magnetometer (e.g., the magnetometer 104 shown in FIGS. 1 and 2) and accelerometer (e.g., the accelerometer 106 shown in FIGS. 1 and 2) according to different sampling rates to reduce the frequency of measurements and shut off some measurements when not needed. Optional aspects are illustrated in dashed lines.

As shown in FIG. 3, the method 300 begins in step 302 by powering up the system (e.g., system 100 shown in FIGS. 1 and 2). In step 304, the method 300 includes an initial assumption or condition that the subject is awake. Based on the assumption that the subject is awake and, as such, at step 304, the subject will most likely be causing body motion artifact that may be tracked and detected using relatively slow sampling with the accelerometer, since a periodic check would indicate that the subject is drastically changing body orientations. The accelerometer is controlled according to the first sampling rate (e.g., one sample in the range of every 2 to 10 seconds). The data signal from the accelerometer at the first sample rate corresponds to the positional body status of the subject. The sampled data signal is filtered by an envelope detector and a low pass filter. The low pass filter has a cutoff frequency in the range of about 0.1 to 2.0 Hz. The filtered data signal from the accelerometer at the first sampling rate can be analyzed to determine the body orientation of the subject, such as vertical or recumbent.

In the next step 306, the method 300 includes determining if the subject is vertical, based on the filtered accelerometer data signal from step 304. Based on a determination that the subject is vertical, the method returns to step 304. Based on a determination that the subject is in a recumbent body orientation (e.g., not vertical), then the method 300 proceeds to step 308. In some aspects, the recumbent body orientation may also correspond to a prone position, a supine position, or a lateral position of the subject.

Next in step 308, based on a determination that the subject is in a recumbent body orientation, the method 300 includes controlling the magnetometer at the first sampling rate. The data signal from the magnetometer at the first sampling rate corresponds to the positional body status of the subject. The sampled data signal is filtered by an envelope detector and a low pass filter. The low pass filter has a cutoff frequency in the range of about 0.1 to 2.0 Hz. The filtered data signal from the magnetometer at the first sampling rate can be analyzed to determine the body direction of the subject, such as whether the subject is motionless. The method 300 proceeds to step 310.

Next in step 310, the method 300 includes determining whether the body orientation of the subject has changed within a predefined time period (e.g., 3 minutes). Based on a determination that the body orientation of the subject has changed within the predefined time period, the method 300 includes returning to controlling the magnetometer at the first sampling rate, in step 308. The filtered data signal from the magnetometer at the first sampling rate can be analyzed to determine the body direction of the subject, such as whether the subject is motionless. Based on a determination that the body direction of the subject has not significantly changed within the predefined time period (e.g., is therefore still or motionless), the method 300 proceeds to step 312. Based on a determination that the body direction of the subject has changed within the predefined time period, then the method 300 returns to step 308.

In step 312, the method 300 includes controlling the accelerometer according to a second sampling rate (e.g., in the range of about 1 to 10 Hz). If the body orientation of the subject has been determined in step 310 to be substantially motionless within a predefined time period, then presumably the subject is sleeping. The data signal from the accelerometer at the second, faster, sampling rate corresponds to the respiration of the subject. The sampled data signal is filtered by a bandpass filter with a passband in the range of 0.1 to 2 Hz. The filtered data signal from the accelerometer at the second sampling rate can be analyzed to determine the respiration of the subject. The method 300 proceeds to step 314.

In step 314, the method 300 includes fusing measurement signal from the accelerometer and magnetometer to obtain body heading information. The body heading information enables the method 300 to distinguish inspiration from expiration in different orientations. The filtered data signal from the accelerometer, which was sampled at the second, faster sampling rate may be down sampled or decimated to the first sampling rate. The down sampled accelerometer signal and the magnetometer signal at the first sampling rate can then be analyzed to determine body heading. The method 300 proceeds to step 316.

In step 316, the method 300 includes determining whether the filtered accelerometer data signal at the second sampling rate corresponding to respiration is a strong signal. In some aspects, a strong signal is considered to be a respiration signal that may be easily extracted from background noise. For instance, if the signal-to-noise ratio was greater than a threshold (e.g., a threshold of 2) then the signal may be considered strong. Based on a determination that the accelerometer signal is not strong, then the method 300 proceeds to step 318. Based on a determination that the accelerometer data signal is a strong signal, then the method 300 proceeds to step 318.

In step 318, the method 300 includes turning on a gyroscope and controlling the gyroscope according to the second sampling rate. The gyroscope data signal corresponds to the body direction changes of the subject. In some aspects, the gyroscope data signal is filtered by a bandpass filter with a passband in the range of 0.1 to 2 Hz and corresponds to the respiration of the subject. The method 300 proceeds to step 320.

In step 320, based on a determination that the accelerometer signal is a strong signal in step 316, then the method 300 includes determining whether the respiration signal is regular (e.g., periodic or pseudo-periodic). Based on a determination that the subject is determined to exhibit regular respiration signals, then there is a strong presumption that the subject may be snoring or exhibiting physical movements that are involved with sleeping and the method 300 proceeds to optional steps 322 and 324. Based on a determination that the respiration of the subject is not regular, then the method 300 proceeds back to step 316. If optional steps 322 and 324 are not performed, then the method 300 proceeds to step 326.

In optional step 322, the method 300 includes checking any other available sleep indicators. For instance, the respiration rate may be measured using sound captured from a microphone since a regular respiration rate may be an indication of sleep state. The method 300 then proceeds to optional step 324.

In optional step 324, the method 300 may include determining whether available sleep indicators signify sleep. Based on a determination that the sleep indicators do not signify sleep, then the method 300 returns to step 322 to check any other available sleep indicators. Based on a determination that the sleep indicators signify sleep, then the method 300 proceeds to step 326.

In step 326, the method 300 includes controlling the accelerometer according to a second sampling rate (e.g., fast) and controlling the magnetometer according to a first sampling rate (e.g., slow) based on a detection that the subject is asleep. The accelerometer data signal is filtered as in step 312 to provide respiration signal for the subject. The magnetometer data signal is filtered as in step 308 to provide body direction signal for the subject. The method 300 then proceeds to step 328.

In step 328, the method 300 includes using the filtered accelerometer data signal, which is relatively smaller amplitude signal to detect respiration. The down sampled and filtered accelerometer data signal, such as in step 314 which corresponds to body orientation and the filtered magnetometer data signal, as in step 308, which corresponds to body direction are analyzed to detect large subject body motions. The method 300 proceeds to step 330.

In step 330, the method 300 includes using the data signals generated in step 328 to determine whether the subject has moved in some substantial way and may not be sleeping. Based on a determination that the subject did not move in a substantial way (and is therefore sleeping), the method 300 returns to step 326. Based on a determination that the subject does not move (e.g., the subject is continues to sleep without substantial movement), then, the method 300 continues to generate respiration signal and also inspiration and expiration signal while in the loop consisting of steps 326, 328 and 330. The inspiration signal can then be used by an implantable stimulation system for determining when to generate electrical stimulation signals to one or more nerves which innervate an upper airway muscle of a subject to reduce or prevent the incidence of obstructive sleep apnea (OSA). Based on a determination that the subject has moved (e.g., and is determined to no longer be sleeping), then the method 300 proceeds to step 332.

In step 332, based on the determination that the subject has moved in step 330, then the method 300 includes resetting all the signal processing filters used in the method 300. When the digital data signal from the accelerometer and magnetometer is processed by a digital filter, then resetting the digital filters includes a reset to their internal initial states. The method 300 proceeds to step 334.

In step 334, the method 300 includes determining whether the subject is vertical. Based on a determination that the subject is determined to be vertical, then the subject is awake (e.g., not sleeping) and the method 300 returns to step 306. In step 306, the method 300 includes sampling body status at the first sampling rate with the accelerometer may converse energy by making more infrequent measurements since the subject's motions will be presumed to be motion artifact. Based on a determination that the subject is not vertical, then the method 300 returns to determining whether the respiration signal is regular in step 320.

It should be noted that the specific time periods and the specific measurement frequencies are mentioned above are intended to be illustrative purposes only, and they should not be understood or interpreted as being limited to those specific listed time periods and/or measurement frequencies.

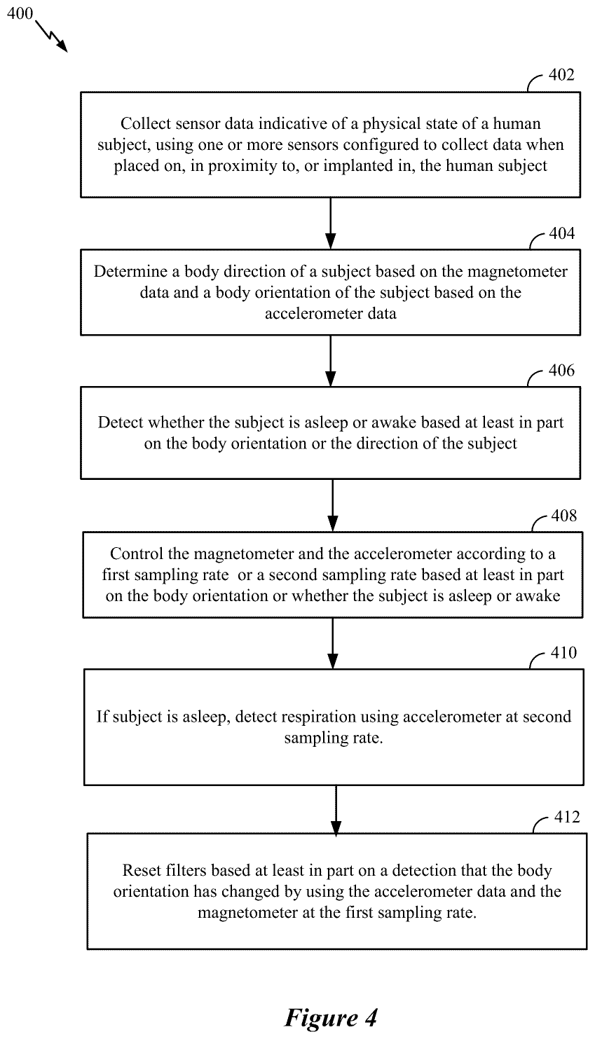

FIG. 4 is a conceptual flowchart summarizing a method for resetting the signal filters based on using a magnetometer and an accelerometer to detect whether a subject is awake or asleep using the systems described herein, and optionally adjusting OSA stimulation based on the same. According to various different aspects, one or more of the illustrated blocks of method 400 may be omitted, transposed, and/or contemporaneously performed. The method allows a system (e.g., the system 100 shown in FIGS. 1 and 2) to reset digital filters based on a detection of whether the subject is awake or asleep using measurement signal from a magnetometer (e.g., the magnetometer 104 shown in FIGS. 1 and 2) and an accelerometer (e.g., the accelerometer 106 shown in FIGS. 1 and 2).

At block 402, the system 100 may begin with the collection of measurement signal (or sensor signal) indicative of a physical state of a human subject using at least a magnetometer to obtain magnetometer signal and an accelerometer configured to obtain accelerometer signal.

In some aspects, the magnetometer may be further configured to obtain the magnetometer signal when the subject is in a vertical position. In some aspects, the magnetometer may be further configured to obtain the accelerometer signal when the subject is in a recumbent position, as in a prone position, a supine position, or a lateral position.

At block 404, the controller (e.g., controller 102 shown in FIGS. 1 and 2) may be configured to determine a body direction of a subject based on the magnetometer signal and a body orientation of the subject based on the accelerometer signal.

At block 406, the controller may be further configured to detect whether the subject is asleep or awake based at least in part on the body orientation or the direction of the subject. In some aspects, the system may include a gyroscope configured to obtain periodic gyro measurements and the controller may be further configured to cause the gyroscope to initiate obtaining the periodic gyro measurements based at least in part on a determination of a lack of body orientation change by the subject within the time period. In some aspects, the accelerometer may be further configured to obtain periodic accelerometer measurements and the controller may be further configured to: cause the accelerometer to initiate obtaining the periodic accelerometer measurements based at least in part on a determination of a lack of body orientation change by the subject within a time period.

In some aspects, the controller may be further configured to determine whether there is a lack of body orientation change by the subject within a time period. In some aspects, the controller may be further configured to: in response to the system being powered on, control the accelerometer according to the first sampling rate based at least in part on a detection that the subject is awake.

At block 408, the controller is further configured to control the magnetometer and the accelerometer according to a first sampling rate or a second sampling rate based at least in part on the body orientation or whether the subject is asleep or awake. In some aspects, the controller may be further configured to: control the magnetometer according to the first sampling rate based at least in part on the body orientation of the subject being in a recumbent position and a determination that there is a lack of body orientation change by the subject within the time period; and generate heading information based at least in part on the direction of the subject obtained from the accelerometer and the body orientation of the subject obtained from the magnetometer.

In some aspects, the system further comprises a gyroscope configured to obtain periodic gyro measurements and the controller may be further configured to: cause the gyroscope to power on and control the gyroscope according to the second sampling rate based at least in part on a determination that a respiration signal does not exceed a threshold. The respiration signal may be obtained based at least in part on using the accelerometer.

In some aspects, the controller may be further configured to: control the accelerometer according to the second sampling rate and control the magnetometer according to the first sampling rate based at least in part on a detection that the subject is asleep and a determination that a respiration signal is periodic. The respiration signal may be obtained based at least in part on using the accelerometer.

At block 410, the controller may be further configured to: control the accelerometer according to the second sampling rate and control the magnetometer according to the first sampling rate further based at least in part on obtaining indicators that indicate that the subject is asleep.

At block 412, the controller may be further configured to: after detecting that the subject is asleep, reset digital filters based at least in part on a detection that the body orientation has changed by using the accelerometer controlled according to the second sampling rate to detect a respiration event and the magnetometer controlled according to the first sampling rate to detect movement of the subject.

In some aspects, the controller may be further configured to: after resetting the signal filters, control the magnetometer according to the first sampling rate based at least in part on a determination that the body orientation is vertical and a determination that the body orientation is a recumbent position. In some aspects, the controller may be further configured to: after resetting the digital signal filters, control the accelerometer according to the second sampling rate and control the magnetometer according to the first sampling rate based at least in part on: (a) a determination that the body orientation of the subject is in a recumbent position, such as in a prone position, a supine position, or a lateral position, (b) a determination that a respiration signal is periodic, and (c) a detection that the subject is asleep.

In some aspects, the controller may be further configured to: detect an inspiration and an expiration by the subject based at least in part on information obtained by the accelerometer with respect to a chest of the subject. The information may be determined based at least in part on measurements obtained from the accelerometer or the magnetometer. In some aspects, the inspiration and expiration by the subject may be detected based at least in part on heading information obtained from the magnetometer. In some aspects, the controller may be further configured to: obtain an absolute orientation of the subject based at least in part on a coordinate frame relative to a reference frame. The coordinate frame may be obtained based at least in part on the accelerometer and the reference frame is obtained based at least in part on the magnetometer. In some aspects, the body orientation of the subject with respect to magnetic north may be based at least in part on the direction of the subject obtained by the accelerometer. In some aspects, the controller may be further configured to: perform re-calibration of the body orientation of the subject with respect to a magnetic north based at least in part on the body orientation change within the time period.

It is understood that the methods illustrated by FIGS. 3-4 are exemplary in nature and that the steps described herein may be combined to generate alternative embodiments.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A system for determining respiration of a subject, the system comprising:
an accelerometer configured to sense body status of the subject at a first sampling rate and generate a first accelerometer signal;
a controller coupled to the accelerometer and configured to control sampling rates of the accelerometer; and
a first filter comprising an envelope detector and a low pass filter, wherein the first filter is configured to process the first accelerometer signal and generate a body orientation signal corresponding to a body orientation of the subject, wherein the controller is further configured to determine if the body orientation of the subject is recumbent based on the body orientation signal.

2. The system of claim 1, further comprising:
a magnetometer coupled to the controller and configured to sense the body status of the subject and generate a magnetometer signal at the first sampling rate, wherein the first filter is further configured to process the magnetometer signal and generate a body direction signal corresponding to a body direction of the subject, wherein the controller is further configured to determine if the subject is motionless based on the body direction signal.

3. The system of claim 2,
wherein the accelerometer is further configured to sense body movement at a second sampling rate and generate a second accelerometer signal, wherein the second sampling rate is greater than the first sampling rate, and further comprising:
a second filter comprising a bandpass filter, wherein the second filter is configured to process the second accelerometer signal and to generate a respiration signal corresponding to the respiration of the subject, wherein the controller is further configured to determine if the respiration signal is regular.

4. The system of claim 3, wherein the controller is further configured to determine a body heading signal of the subject based at least in part on processing the body orientation signal and the body direction signal.

5. The system of claim 4, wherein the controller is further configured to determine that the subject is asleep based at least in part on processing the respiration signal and at least one of the body orientation signal, the body direction signal, and the body heading signal.

6. The system of claim 4, wherein the controller is further configured to determine inspiration and expiration phases of the respiration of the subject based at least in part on processing the respiration signal and at least one of the body orientation signal, the body direction signal, and the body heading signal.

7. The system of claim 6, further comprising:
a stimulation system for generating nerve stimulation signals; and
a lead coupled to the stimulation system and comprising a neural interface at its distal end, wherein the controller is coupled to the stimulation system and further configured to control generating the nerve stimulation signals during the inspiration phase of the respiration of the subject.

8. The system of claim 1, wherein the first sampling rate is one sample in a range of every 2 to 10 seconds.

9. The system of claim 3, wherein the second sampling rate is in a range of 1 to 10 Hz.

10. The system of claim 1, wherein the low pass filter has a cutoff frequency in a range of 0.1 to 2.0 Hz.

11. The system of claim 3, wherein the bandpass filter has a passband within a range of 0.1 to 2.0 Hz.

12. The system of claim 3, further comprising:
a gyroscope coupled to the controller and configured to sense body direction change and generate a gyroscope signal at the second sampling rate, wherein the second filter is further configured to process the gyroscope signal to provide a body direction change signal corresponding to the body direction change of the subject,
wherein the controller is further configured to determine that the respiration of the subject is regular based on the body direction change signal.

13. The system of claim 4, wherein the controller is further configured to determine that the subject is vertical based on at least one of the body orientation signal, body direction signal, and body heading signal,
wherein the first and second filters comprise digital filters having internal initial states; and, wherein the controller is further configured to reset the internal initial states of the first and second filters to initial states.

14. A method for determining respiration of a subject, the method comprising:
sensing, using an accelerometer, body status of the subject at a first sampling rate in a range of every 2 to 10 seconds;
generating, using the accelerometer, a first accelerometer signal;
processing the first accelerometer signal with a first filter and generating a body orientation signal, wherein the first filter comprises an envelope detector and a low pass filter, wherein the low pass filter comprises a cutoff frequency in a range of 0.1 to 2.0 Hz; and
determining if a body orientation is recumbent based on the body orientation signal.

15. The method of claim 14, further comprising:
sensing, using a magnetometer, the body status of the subject at the first sampling rate;
generating, using the magnetometer, a magnetometer signal;
processing the magnetometer signal with the first filter;
generating a body direction signal; and
determining if the body status of the subject is motionless based on at least one of the body orientation signal or the body direction signal.

16. The method of claim 15, further comprising:
sensing, using the accelerometer, body motion of the subject at a second sampling rate in a range of 1 to 10 Hz;
generating, using the accelerometer, a second accelerometer signal;
generate a respiration signal by processing the second accelerometer signal with a second filter, wherein the second filter comprises a bandpass filter with a passband within a range of 0.1 to 2.0 Hz; and
determining if the respiration signal is regular based on the respiration signal.

17. The method of claim 16, further comprising:
generating a body heading signal by processing the body orientation signal with the body direction signal; and
determining if the subject is asleep based on at least one of the body orientation signal, the body direction signal, and the body heading signal.

18. The method of claim 16, further comprising:
determining inspiration and expiration phases of the respiration of the subject based on the respiration signal.

19. The method of claim 18, further comprising:
generating, using a stimulation system, nerve stimulation signals;
providing a lead coupled to the stimulation system and having a neural interface at its distal end; and
generating nerve stimulation signals during the inspiration phase of the respiration of the subject.

20. The method of claim 14, further comprising:
sensing, using a gyroscope, body direction change and generating a gyroscope signal at a second sampling rate;
processing the gyroscope signal with a second filter comprising a bandpass filter with a passband within a range of 0.1 to 2.0 Hz;
providing a body direction change signal corresponding to the body direction change of the subject; and
determining that the respiration of the subject is regular based on the body direction change signal.

21. The method of claim 17, further comprising:
determining that the subject is vertical based on at least one of the body orientation signal, body direction signal, and body heading signal; and
resetting internal initial states of the first and second filters comprising digital filters to their initial states.
